(19) United States
(12) Patent Application Publication
(10) Pub. No.: US 2003/0166916 A1
(43) Pub. Date: Sep. 4, 2003

(54) Method for purifying nucleic acids

(76) Inventors: Thomas Kolzau, Hamburg (DE); Heinz Gerard Kohn, Hamburg (DE); Wilhelm Pluster, Hamburg (DE); Mathias Ulbricht, Berlin (DE)

Correspondence Address: RANKIN, HILL, PORTER & CLARK, LLP, 700 HUNTINGTON BUILDING, 925 EUCLID AVENUE, SUITE 700, CLEVELAND, OH, 44115-1405, US

(21) Appl. No.: 10/182,957

(22) PCT Filed: Feb. 2, 2001
(86) PCT No.: PCT/EP01/01134

(30) Foreign Application Priority Data
Feb. 11, 2000 (DE) 10006591.0

(51) Int. Cl. C07H 21/02; C07H 21/04

(52) U.S. Cl. 536/25.4

(57) ABSTRACT

The invention relates to a method for purifying nucleic acids contained in a raw material using a porous membrane, the raw material possibly being prepurified and then mixed with a binding buffer containing a precipitant at a concentration such that the final concentration of the mixture of raw material and binding buffer it shall exceed the concentration required to precipitate nucleic acids, the raw material containing the nucleic acids being allowed to permeate the membrane in a manner that the nucleic acids shall be selectively retained at the membrane's surface or in its pores, the membrane being washed if called for and optionally the bound nucleic acids being eluted again from the membrane in a further step.

The present invention relates to a method using a porous membrane for purifying nucleic acids contained in a raw material.

A considerable number of procedures already are extant wherein, if called-for after a pre-purification stage, a raw material containing nucleic acids is aspirated or pressed through a porous membrane in a manner that, while the raw material crosses the membrane, said nucleic acids shall be selectively bound to said membrane's surface.

As a rule nucleic-acid affinity for the membranes is attained by using a special configuration of the membrane surface jointly with specific conditions in the buffer medium wherein the raw material crosses the membrane.

Illustratively a procedure is known from U.S. Pat. No. 5,438,128 whereby special polymer membranes functionalized with ion-exchange groups are used to purify nucleic acids. To attain selective binding, the raw material containing the nucleic acids is received in a buffer of low ionic concentration and while in this buffer is aspirated or forced through the membrane while selective binding takes place.

Another known procedure is known from the European patent document 0 512 767. In this known procedure, the nucleic acids are bound to hydrophilic membranes, an organic solvent such as iso-propanol being added in a high concentration to the binding buffer system.

The objective of the present invention is to purify nucleic acids by a method which shall be easier to carry out than the known procedures This goal is attained by the method defined by the features of claim 1 and of claim 5.

The invention shall cover two modes of implementation. In the first mode, arbitrary membranes may be used. Special membranes shall be used in the second mode.

In the first mode of implementation of the present invention, the raw material, which may have been pre-purified, is mixed with a binding buffer containing polyethylene glycol (PEG) and/or at least a salt in such a concentration that the final concentration of the mixture of raw material and binding buffer shall be above that value required to precipitate nucleic acids, and then permeating the membrane with the mixture of raw material and binding buffer for instance under vacuum or by centrifuging, the nucleic acids being selectively retained at the surface or in the pores (deep filtering effect) of said membrane.

The membrane then may be washed in a further stage to remove any unspecifically bound contaminants. If desired, said washing stage may be followed by an elution stage wherein the bound nucleic acids then are again detached from the membrane.

In especially preferred manner, PEG may be used as the precipitating agent in a final concentration of more than 6% relative to the mixture of binding buffer and raw material.

The purification of nucleic acids, in the presence of PEG 8000, at silica beads is described in Engelstein et al, Microbial & Comparative Genomics, vol. 3, #4,1998. The binding buffer used in that publication contains 20% PEG 8000. In this known method said PEG 8000 is intended to replace the chaotropic reagents required, up to that time, in purifying nucleic acids at silica substrates. Said document in no way either describes or indicates that the mechanism it discloses also might be feasible with substrates other than silica and also substrates manufactured into other shapes than beads.

Furthermore the initially cited European patent document 0 512 767 states in its table on page 7 that, in the presence of 10% of 20% PEG, binding of DNA does not take place at membranes: this statement is in conflict with applicant's results.

Typically plastic membranes for instance made of polypropylene, polyamides, polyesters, polysulfone or PVDF are used.

Preferably the pores of the membranes shall be 0.2 to 10 p in diameter, yield being optimized by selecting the pore diameter. As the pore diameter decreases, the membrane's retention rate increases. On the other hand smaller pores also clog more rapidly, and therefore the pore diameter may not be too small.

Preferably the membranes are designed in a manner to attain both surface filtering and filtering in depth.

The selected binding conditions moreover shall intrinsically assure that the nucleic-acid shall be present in squashed form to allow optimal retention at and in the membrane.

In the second mode implementing the invention, the membranes used in purification are functionalized with deprotonatable groups, especially with groups of sulfonic acid, carboxylic acid or phosphoric acid.

In this variation of the invention, again the raw material containing the nucleic acids is contained in a binding buffer and made to pass through the membrane which in this instance was functionalized, the binding buffer containing a precipitant for nucleic acids. Thereupon and as called for, the membrane may be washed and the nucleic acids next can then be separated from the membrane.

In the invention, the precipitant in the mixture of binding buffer and raw material shall be at a concentration above the magnitude required to precipitate nucleic acids. In this preferred implementation of the invention, the nucleic acids are precipitated in the presence of the binding buffer. If thereupon the mixture of binding buffer and raw material is aspirated through the membrane, the nucleic acids shall remain adhering to the membrane.

Illustrative precipitants are salt, PEG and also isopropanol, which are a few among the many examples suitable for the invention.

As regards a further known procedure disclosed in U.S. Pat. No. 5,705,628, the nucleic acids are bound in the presence of PEG to magnetic particles having functionalized surfaces. The procedure discussed in said US patent is specifically designed for magnetic beads which, compared to the membranes of the present invention, entail more complex handling. Beads moreover require larger elution volumes than do membranes.

It was found that methods comprising the functionalized membranes of the invention allow very good nucleic-acid yields, especially when, in the manner of another preferred embodiment of the invention, the functionalized groups are bound to polymer chains of which one of the ends are fixed to the membrane surface and the other ends are freely displaceable. These polymer chains substantially boost the retaining properties of membranes for nucleic acids.

Depending on the ambience, the polymer chains will assume different positional attitudes relative to the membrane. If the pH value is rather alkaline or neutral and/or at lower ionic concentrations, the polymer chains will rise fairly straight from the membrane. If the pH value is lowered and/or the ionic concentration is raised, the polymer chains will rest parallel to and against the membrane.

It was found in the present instance that the highest retention rate of membrane with respect to nucleic acids shall be attained when on one hand the polymer chains shall stand fairly stretched away from the membrane though not being entirely straight (tentacle structure). Under such conditions—which shall be present when in the case for instance of higher ionic concentrations or of higher PEG concentration—the nucleic acid molecule also assumes a squashed shape in which it shall be retained in especially effective manner when in its tentacle shape.

Said polymer chains for instance may be polyacrylic acids or the like.

In this embodiment variation also the membranes may exhibit pores preferably 0.2 to 10μ in diameter, the yield being optimized by selecting the appropriate pore diameter.

In both cases elution of the nucleic acids adhering to the membrane may be carried out using buffers of low ionic concentration or water at room temperature. To elute, the used buffer is aspirated or forced through the membrane. Any washing stage between binding and elution may be carried out in the same way for instance using ethanol or the like.

Preferably those conditions shall be set for elution under which the polymer chains shall be as stretched as possible while configured away from the membrane and the nucleic acid molecule as well shall be straight. Under these conditions, which for instance are those for elution, the nucleic acid molecular may be detached especially well from the membrane.

In principle all buffers or mixtures may be aspirated under vacuum through the membrane or be forced through it for instance by centrifuging.

Preferably the method of the invention shall be carried out using membranes configured within microtitration filtering trays, spin columns or reaction containers.

The invention is elucidated below in illustrative manner.

A: Manufacturing Functionalized Membranes

### EXAMPLE 1

(a) Modifying Polypropylene Membranes

A polypropylene microfiltration membrane (Accurel 2E HF, nominal pore size=0.2μ, membrane thickness 150μ, Membrana GmbH, Wuppertal [Germany]; or Test Specimen #1333-12A, nominal pore size=0.45μ, membrane thickness 110μ, 3M, St. Paul, USA) of diameter d=80 mm is equilibrated after 2 h-agitation with 100 mM solution of benzophenone in acetone. Thereupon the membrane is coated with a 10% aqueous acrylic-acid solution. Next it is illuminated for 15 min with uv (UVA-Spot 200; Dr. Honle GmbH, Planegg [Germany]). Finally the modified membrane is extracted with water for 24 h at 60° C. and dried.

(b) Modifying Nylon Membranes

A nylon microfiltration membrane (Schleicher & Schull, nominal pore size=0.45 p, membrane thickness=127μ) is modified under the same conditions as in (a).

B: Purifying Nucleic Acids

The surface-modified membranes prepared in the manner of Examples 1 (a) and 1(b) above were placed in a 96-microfiltration tray when carrying out the purification below.

### EXAMPLE 2

To determine the modified membranes=retention abilities regarding pDNA, 1 μg of pDNA was mixed each time with 200 μltr binding buffer (BB), namely, BB1=5 mM tris, pH=7.5; BB2=4M NaCl, 10% PEG 8000, pH=4.6. Purification is by centrifuging at 3,000 rpm according to the bind/wash/elute principle. The basic material of this test is surface-modified PP of 0.45μ pore width. Following incubation, washing with 200 μltr of 70% EtOH is carried out twice. Elution is carried out with 30 μltrs tris-HCl (5 mM, pH=7.5). Lastly elution is carried out a second and last time using 100 μtr. Semi-quantitative quantitative analysis of the 4 μltr eluate is carried out using ethidium bromide gel electrophoresis (omitted).

Whereas binding to the membrane will not take place when using the binding buffer BB1, when using the binding buffer BB2 on the other hand it was possible to nearly quantitatively bind the PDNA to the membrane and to elute it again during the first step of elution.

### EXAMPLE 3

pDNA purification is carried out by the principle of alkaline lysation. For that purpose bacteria are centrifugally removed from 1.5 mltr of a bacterial culture and mixed with the buffers B1-B3 (B1: 100 μltr; B2=300 μltr and B3=300 μltr) from the “Perfect Prep Plasmid” kit of Eppendorf Co. After centrifuging, the clear lysate is mixed each time with 700 μltr of binding buffer (see Table 1). The test specimen is processed under the conditions cited in Example 1. The control used is in the form of a plasmid preparation, the entire purification procedure being carried out by means of the “Perfect Prep Plasmid” kit of Eppendorf Co. and from an identical 1.5 mltr of bacterial culture (data omitted). As a further control, the mixture of clarified lysate and the particular binding buffer (see Table 1) is centrifuged at room temperature for 30 min at 12,000 g and the supernatant is removed. This is followed by washing twice with 200 μltr of 70% ethanol, drying under vacuum and adding 30 μltr of tris-HCl, pH=7.5 (data omitted).

The yield and purity were determined by photometry and analysis by using ethidium bromide gel electrophoresis (Table 1; FIG. 1). Said Table lists the concentration of eluted pDNA in ng/μltr using the Eppendorf photometer (“BioPhotometer”).

TABLE 1

| | Binding buffer: | Binding buffer: | Binding buffer: |
|---|---|---|---|
| | 20% PEG | 20% PEG | 20% PEG |
| | (MW = 8,000) | (MW = 8,000) | (MW = 8,000) |
| | pH = 3.5 | pH = 4.6 | pH = 7.4 |
| Nylon, 0.45μ | 9 ng/μltr | 4 ng/μltr | 23 ng/μltr |
| unmodified | | | |
| (S & S) | | | |
| Nylon, 0.45 μltr | 16 ng/μltr | 102 ng/μltr | 142 ng/μltr |
| coated with | | | |
| acrylic acid | | | |

The tracks of the gel shown in FIG. 1 were loaded as follows:

1) binding buffer, pH 7.4, modified membrane

2) binding buffer, pH 4.6, modified membrane

3) binding buffer, pH 3.5, modified membrane

4) binding buffer, pH 7.4, unmodified membrane

5) binding buffer, pH 4.6, unmodified membrane

6) binding buffer, pH 3.5, unmodified membrane.

Both the gel and photometric analyses concurred that, with respect to the modified membranes, PDNA may be purified at pH 4.6 and pH 7.4 at high concentrations, whereas as regards the unmodified membrane only a low yield in purified pDNA was found at pH 7.4.

[image]

1. A method for purifying nucleic acids contained in a raw material, using a membrane, wherein
the raw material—which may have been prepurified—is mixed with a binding buffer containing PEG and/or at least one salt in a concentration such that the resultant final concentration in the mixture of raw material and binding buffer shall be above the value required to precipitate nucleic acids,
the binding-buffer containing raw material is made to permeate the membrane, the nucleic acids being selectively retained at the surface or in the pores of the membrane,
the membrane shall be washed if called for,
and, if called for, the bound nucleic acids are eluted again from the membrane.

2. Method as claimed in claim 1, characterized in that PEG is present in a final concentration >6%.

3. Method as claimed in one of the above claims, characterized in that the membrane is made of plastic.

4. Method as claimed in claim 5, characterized in that the membrane consists of polypropylenes, polyamides, polyesters, polysulfones, PVDF etc.

5. Method for purifying nucleic acids contained in a raw material, using a porous membrane functionalized with deprotonatable groups, wherein
the raw material—which may have been prepurified—is mixed with a binding buffer containing a precipitant for nucleic acids,
the mixture of raw material and binding buffer is allowed to permeate the membrane, the nucleic acids being selectively retained a the membrane's surface or in its pores,
if called for, the membrane is washed,
and, if called for, the bound nucleic acids are eluted again from the membrane in a further step.

6. Method as claimed in claim 5, characterized in that the mixture of binding buffer and raw material exhibits a final concentration of precipitant which is larger than the value required to precipitate nucleic acids.

7. Method as claimed in either of claims 5 and 6, characterized in that the precipitant is PEG.

8. Method as claimed in claim 7, characterized in that the final PEG concentration >6%.

9. Method as claimed in one of claims 5 through 8, characterized in that the membrane is made of plastic.

10. Method as claimed in claim 9, characterized in that the membrane is made of polypropylenes, polyamides, polyesters, polysulfones, PVDF etc.

11. Method as claimed in one of claims 5 through 10, characterized in that the membrane is functionalized with groups of sulfonic acid, carboxylic acid or phosphoric acid.

12. Method as claimed in one of claims 5 through 11, characterized in that the groups are bound to polymer chains of which one end is affixed to the membrane surface and the other end is freely displaceable.

13. Method as claimed in claim 12, characterized in that the affixed polymer chain is an acrylic acid.

14. Method as claimed in one of the above claims, characterized in that the membrane's pores are 0.2 to 10μ in diameter.

15. Method as claimed in one of the above claims, characterized in that the mebrane's thickness <500μ.

16. Method as claimed in one of the above claims, characterized in that the membrane is manufactured for use in micro-titration trays, spin columns or reaction containers.

17. Method as claimed in one of the above claims, characterized in that the binding-, washing- and/or elution-buffer is vacuum-aspirated through the membrane.
